(19) United States
(12) Patent Application Publication
(10) Pub. No.: US 2007/0049984 A1
(43) Pub. Date: Mar. 1, 2007

(54) IMPLANTABLE DEVICE FOR MEASURING BIOMETRIC BLOOD PARAMETERS

(76) Inventor: Peter Osypka, Rheinfelden-Herten (DE)

Correspondence Address: VOLPE AND KOENIG, P.C., UNITED PLAZA, SUITE 1600, 30 SOUTH 17TH STREET, PHILADELPHIA, PA, 19103, US

(21) Appl. No.: 11/463,922

(22) Filed: Aug. 11, 2006

(30) Foreign Application Priority Data
Aug. 31, 2005 (DE) 102005041208.4-35

(51) Int. Cl. A61N 1/00

(52) U.S. Cl. 607/32

(57) ABSTRACT

A device (1) that can be implanted under the outside of a heart (2) for measuring biometric blood parameters, such as, for example, blood pressure, has at least one sensor (3), which can be analyzed telemetrically or by means of transponder technology and which is arranged on or in a housing (4), wherein, a holder for fixing the sensor (3) or its housing (4), for example, in the interior of a heart wall, is also provided. A pin-like anchor (7), which can be inserted into or through the outer wall (5) of the heart (2) and/or the intermediate wall (6) and which is fixed to a pulling element (8) or thread that is connected in the position of use to the sensor (3) or a housing (4) in a suitable way directly or via an opening (13) running in the longitudinal direction. The anchor is formed so that it is held, on one side, in a puncturing cannula (10) and can be moved or inserted, on the other side, with the help of a tool (9) or needle relative to this puncturing cannula (10) and/or through the heart wall (5). In the position of use, a counter anchor (12), for example, a stitch, is provided at the end of the pulling element (15) attached to the opposite end of the housing (4).

### BACKGROUND

The invention relates to a device that can be implanted under the upper or outer heart surface for measuring biometric blood parameters, such as blood pressure, blood composition, pH value of the blood, blood-sugar level, blood temperature, or the like, with at least one sensor that can be analyzed telemetrically or by means of transponder technology, and with a holder for fixing the sensor or a housing containing the sensor on or in the heart.

Such a device is known from EP 1 050 265 A2. Here, an expandable stent, which can be placed in an opening that can be created surgically in the inner wall of the heart, is provided as a holder for a sensor. For this purpose, it is necessary to surgically provide the inner wall of the heart with an opening, which, in addition to the complicated operation, is also disadvantageous for the heart function.

### SUMMARY

Therefore, there exists the objective of creating a device of the type named above, with which the sensor can be mounted on the heart with great security despite the movement of the heart, without negatively affecting the cardiac output.

To achieve this objective, the implantable device defined above comprises a pin-like anchor, which can be inserted from the outside of the heart into or through the outer wall of the heart and/or the intermediate wall, which is fixed to a pulling element or thread, and which is connected to the sensor in the position of use, is provided as a holder for the sensor or its housing, with the thread or the pulling element attaching to the anchor between the two ends of the pin-like anchor, and the anchor can be moved or inserted by means of a tool or needle from the outside into or through the heart wall.

In this way, a device that can be implanted from the outside of the heart is provided having a sensor, which can be mounted within the heart outer wall or the heart intermediate wall or also in the interior of a heart ventricle, which can be performed with precise positioning with the help of the anchor and the pulling element or thread, but which nevertheless causes only very minimal trauma. Simultaneously, this mounting can adapt very well to the movements of the heart. Another advantage is that the sensor can also be removed again relatively easily, in that only the anchor is separated from its pulling element and then the sensor is pulled outwards again at the side facing away from the anchor. A complicated operation and the installation of a stent are avoided.

Here, it is especially preferred when the anchor can pivot relative to the pulling element about its attachment point to this element. Thus, after being placed, the anchor can automatically assume a position perpendicular to the position, through which it was moved or inserted from the outside, so that then the pulling element can already transfer corresponding pulling and retaining forces.

As the attachment point for the tool or the needle to the anchor, a deformation can be provided on the end opposite the piercing tip or the piercing end. The tool or the needle can be coupled detachably to this deformation. Thus, the anchor can be engaged or gripped with a positive fit by the tool or the needle and can be shifted in position in the axial direction.

The anchor can have a reduced cross section for an end of a tool and/or a receptacle opening that is open at this end and that is closed at the opposite end for a tool or needle fitting into this receptacle opening on the side of the anchor at the back in the insertion direction and facing away from the tip at the front end. In this way, the anchor can be shifted into its position of use. Thus, in a simple way, the anchor can be connected to a tool or needle in a positive fit for the implementation.

Here, a puncturing cannula, whose hollow interior corresponds approximately to the outer cross section of the anchor at least at its outer or distal end and which at least partially holds the anchor, is allocated to the device as an aid for the implantation. In this position, the thread or the pulling element runs on the outside of the puncturing cannula. Thus, with this puncturing cannula, the implantation point for the anchor and thus also for the sensor can be prepared, in that the heart wall is punctured accordingly and then the anchor is moved with the pulling element as a holder for the sensor out of this puncturing cannula through the opening created by the cannula and is placed in the position of use.

Preferably, the housing of the sensor has an approximately cylindrical cross-sectional shape with at least one, in particular, two rounded and/or pointed ends, wherein the pulling element attaches to or near one or both ends of the housing at least in the position of use. Thus, the sensor can be placed at nearly any point of the heart wall, within this wall, or also through the heart wall in the interior of a ventricle and is held there by the pulling element attached to it. The cylindrical cross-sectional form simplifies the insertion of this sensor into the opening previously created from the outside by the anchor and the puncturing cannula up to the point, where the sensor is to be placed.

In an advantageous embodiment, the housing of the sensor or the sensor itself can have an opening running in its longitudinal direction, by means of which it can be moved via the thread or the pulling element of the anchor, so that the sensor can be moved into its position of use via the thread after the installation of the anchor. Therefore, first the anchor can be implanted and then the sensor can be moved into its desired position via its pulling element and is fixed there with the help of the pulling element. Here, the movement of the sensor on the pulling element can be tight, so that it is held just by friction on the pulling element, if it is placed in the interior of a ventricle. When moved into a position within the heart wall, the sensor is fixed sufficiently by the surrounding tissue, so that loose movement on the pulling element is also possible.

The end of the pulling element or thread projecting past the sensor on the side facing away from the anchor in the position of use can be provided for holding, for example, against the outside of the heart with a counter anchor or stitch. Here, the simplest counter anchor is a stitch, which the surgeon can attach after inserting the sensor onto the end of the thread or pulling element projecting outside the heart. Similarly, it can also be fixed there by a counter anchor, whereby the pulling element is fixed on one side with the anchor located at the tip and on the other side with the counter anchor, so that the sensor between these anchors is held and remains held in the desired position, even though the heart is moving.

Another preferred configuration of the invention can provide that the sensor which is movable on the pulling element carries an addition thread, by means of which it can also be positioned in the direction opposite the insertion direction and can be pulled out again from the heart.

A modified embodiment can provide that the pulling element with the anchor attaches to one side of the sensor, especially to a rounded or pointed end, and the pulling element for the counter anchor or stitch attaches to the opposite side—in the insertion direction. Thus, the sensor is “carried along” just by implanting the anchor and its pulling element, wherein the anchor is to be placed so that the sensor reaches its provided position, preferably within a ventricle. Then only the second pulling element with the counter anchor or a stitch needs to be provided or mounted.

The housing of the sensor can be composed of biocompatible material and can have a small cross section in relation to its length. Therefore, it can be easily pushed through a previously punctured point of the heart wall, whether during implantation or also later when being removed.

In the housing of the sensor, preferably formed as a pressure sensor, there can be at least two pressure detectors, which are spaced apart relative to each other or which are arranged opposite each other in or on the housing. Such a sensor is suitable primarily for implantation in the intermediate wall of the heart, where it can detect and/or monitor the blood pressure both in the left and also in the right ventricle with the two pressure detectors.

A device for measuring biometric blood parameters is created, above all, by combining individual or several of the previously described features and measures. This device can be implanted and held under the upper or outer surface of the heart easily from the outside, without requiring surgery to create a corresponding holding opening for the holder of the sensor in the intermediate wall of the heart. Instead, the sensor holder essentially composed of a pulling element and an anchor arranged at its free end can be formed by means of a puncturing cannula and a needle in or on the heart wall and with or through this holder, the sensor can be placed and fixed. Here, it can be arranged either in the interior of the heart wall or intermediate wall of the heart or also between the walls defining a ventricle. Due to this simple implantability, which has practically no effect on the heart itself and for which the sensor on the heart has only minimal space requirements, if necessary, several such sensors can also be implanted in order to be able to detect many biometric blood parameters in and on the heart.

### BRIEF DESCRIPTION OF THE DRAWINGS

Embodiments of the invention are described in more detail below with reference to the drawing. Shown in partially considerably schematic representation are:

FIG. 1 is a view of an implantable device for measuring biometric blood parameters in the form of a housing of a sensor and a holder formed as a pulling element or thread, wherein an anchor, which here is pushed within a puncturing cannula for its implantation, is arranged on the end of the pulling element facing away from the sensor;

FIG. 2 is an enlarged scale view of the sensor and the pulling element with an anchor, wherein the anchor is moved transversely relative to the pulling element around its attachment point to the pulling element into the position of use, as it is also provided in the position of use;

FIG. 3 is a view of the tool also indicated in FIG. 1 in the interior of the puncturing cannula when attached to the anchor, by means of which it can be moved out of the puncturing cannula into the position of use after the appropriate point of the heart is punctured;

FIG. 4 is a view of the tip of heart in longitudinal section, wherein the two ventricles and the intermediate wall of the heart can be seen and the puncturing cannula has penetrated the outside of the heart over part of its length;

FIG. 5 is a view corresponding to FIG. 4 after the anchor is pushed out of the puncturing cannula, whereby it comes to lie on the outside of the heart, while the pulling element runs through the channel previously created by the puncturing cannula and a sensor modified relative to FIGS. 1 to 3 is arranged in a housing with an opening, which runs in the longitudinal direction and by means of which the sensor can be moved via the thread or the pulling element of the anchor with the help of a catheter, wherein the sensor is also arranged outside of the heart,

FIG. 6 is a view corresponding to FIGS. 4 and 5 after the sensor has been moved into the interior of the outer wall of the heart, wherein the end of the pulling element opposite the anchor is provided with a counter anchor and fixed;

FIG. 7 is an enlarged scale view of the housing of a sensor with a sensor and an opening running in the longitudinal direction, as well as a pulling element according to FIG. 5 attaching to the housing;

FIG. 8 is a view of a modified embodiment of an implantable sensor with a pressure sensor, for which a pulling element attaches to each of the two ends of the elongated housing, and the pulling element located in front of the sensor in the insertion direction has the anchor, as corresponds approximately to FIGS. 1 and 2′;

FIG. 9 is a view of another embodiment, in which the housing has two opposing pressure sensors and a continuous opening, as well as a pulling element attaching to the rear end in the advancing direction, analogous to the embodiment according to FIG. 7;

FIG. 10 is a view of an embodiment corresponding to FIG. 8, in which the pulling elements are attached to the housing in opposite directions and the pulling element running in the insertion direction has the anchor, wherein the housing contains two sensors; and

FIGS. 11 to 16 are schematic views of further applications which illustrate how and where the sensor can be implanted and fixed with its holder in the form of a pulling element and anchor.

### DETAILED DESCRIPTION OF THE PREFERRED EMBODIMENTS

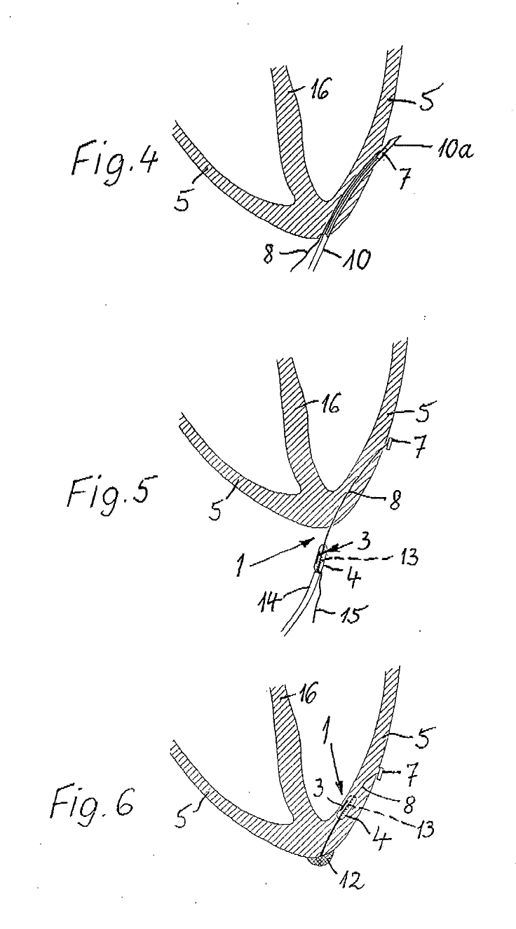

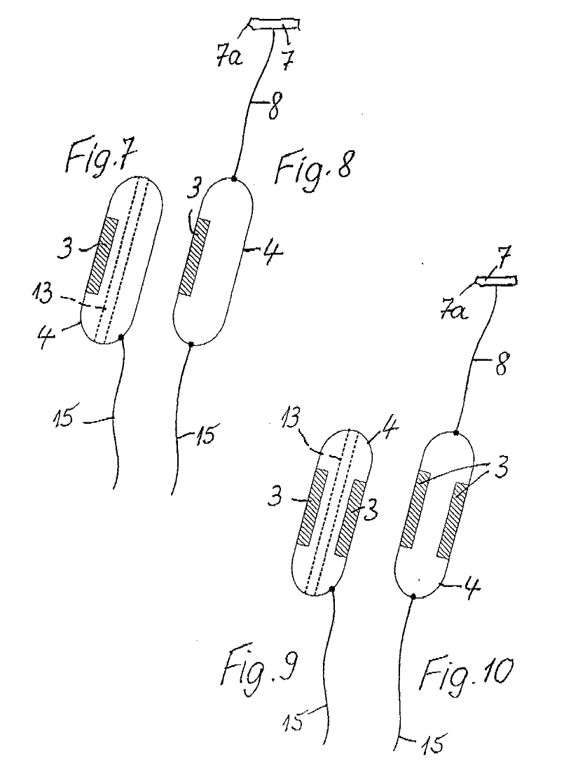

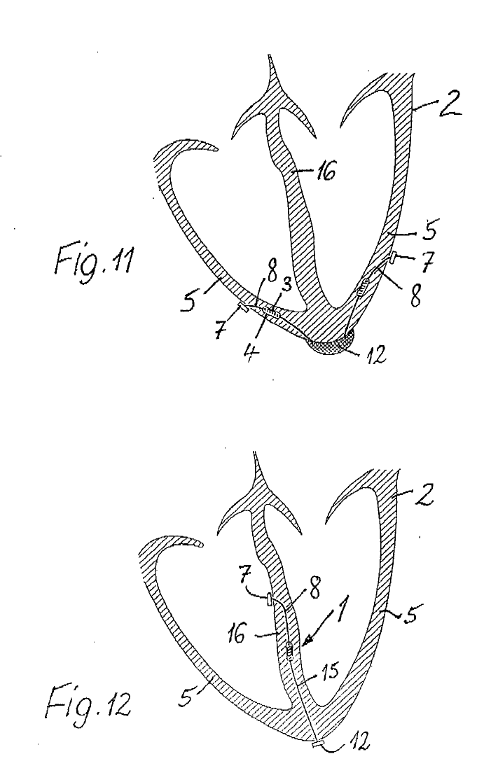

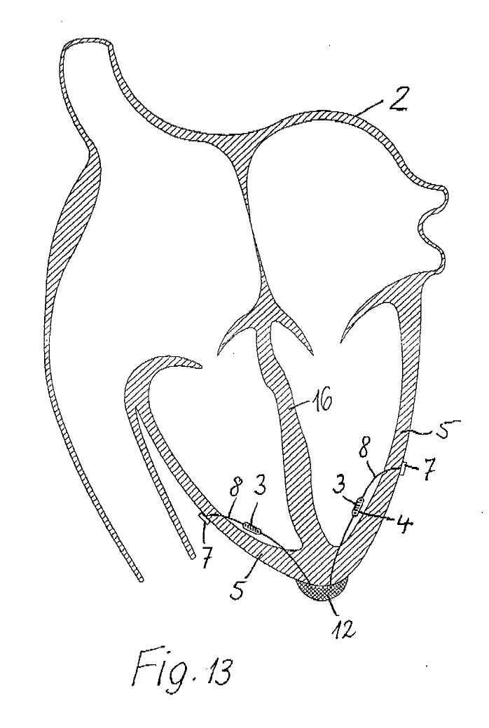

A device, which is designated as a whole with 1 and which can be implanted according to FIGS. 4 to 6 and 11 to 16 from the outside under the upper or outer surface of a heart 2, is used for measuring biometric blood parameters, for example, blood pressure, but also other parameters and has at least one (FIGS. 7 and 8) or two (FIGS. 9 and 10) sensors 3 that can be analyzed telemetrically or by means of transponder technology and a holder to be explained in more detail for fixing each sensor or a housing 4 containing each sensor on or in the heart 2.

A pin-like anchor 7, which can be inserted into or through the outer wall 5 of the heart 2 and/or the intermediate wall 6 of this heart 2 and which is fixed to a pulling element 8 or thread, is provided as a holder for the sensor 3 or its housing 4 in the embodiments. Together this anchor and pulling element form the holder, which is connected to the sensor 3 or to its housing 4 at least in the position of use, as explained in more detail below.

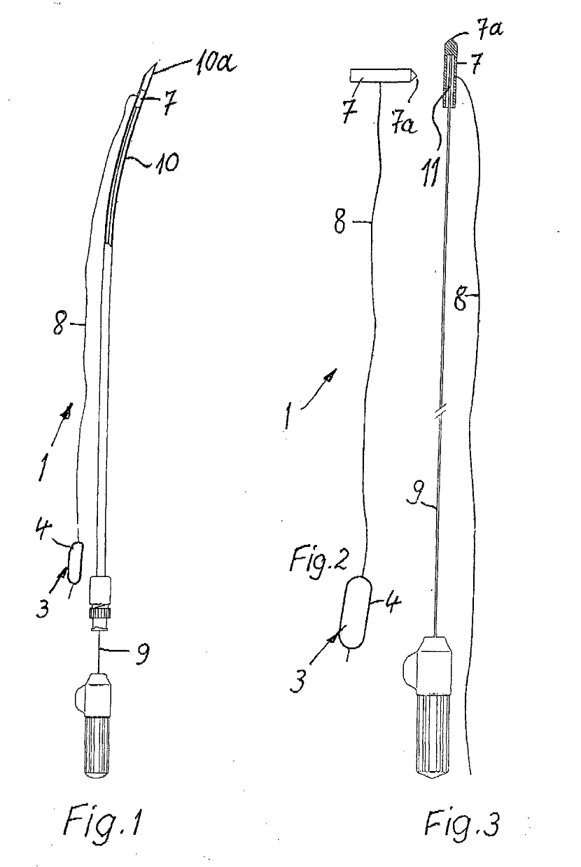

According to FIGS. 1 to 3, the thread or the pulling element 8 attaches to the anchor between the two ends of the pin-like anchor 7, so that it can be set transverse to the pulling element 8 in the position of use analogous to FIG. 2 and thus the position of the pulling element 8 can be fixed, as can be seen clearly, for example, in FIG. 5 and also other drawings of embodiments. The anchor 7, which has a tip 7a at the front end in the insertion direction, can be moved or inserted into or through the heart wall 5 using a tool 9 or needle according to FIGS. 1 and 3. Here, the anchor 7 can pivot relative to the pulling element around the attachment point due to the flexibility of the pulling element 8, as made clear by comparing FIGS. 2 and 3. Here, as an attachment point for the tool or needle 9, on the anchor 7 at its end opposite the piercing tip 7a, there is a deformation, which can be coupled detachably to the tool 9 or needle. In the embodiment, the anchor 7 has, on its side facing away from the tip 7a at the back in the insertion direction, a receptacle opening 11, which is open at the back end and which is closed at the opposite end, for the tool or needle 9 fitting into this receptacle opening, through which the anchor can move into its position of use or can be fixed after being inserted with the help of a puncturing cannula 10 still to be explained when retracting this puncturing cannula 10, in order to finally reach this position of use after also retracting the tool 9.

According to FIG. 1, the already explained puncturing cannula 10, whose hollow interior corresponds to or exceeds at least the outer or distal end 10a approximately of the outer cross section of the anchor 7 and which holds the anchor 7 at least partially or completely according to FIGS. 1 and 4, is contained in the device 1 as an aid for the implantation. In this position, the thread or pulling element 8 runs on the outer side of the puncturing cannula 10.

With the device 1 and here, at first with its puncturing cannula 10, the holder of the sensor 3 or its housing 4, namely the anchor 7 and the pulling element 8 can be transported through the heart wall 5. If the anchor 7 reaches the outside of the heart 2 and the heart wall 5 again, the tool 9 is clamped in this position, but the puncturing cannula 10 is retracted, so that now the anchor 7 is placed on the outside of the heart 2, while the pulling element 8 returns through the heart wall 5. If the housing 4 of the sensor is at the correct distance to the anchor 7 according to FIGS. 1 and 2, the anchor is then located together with the pulling element 8 also within the heart wall 5. Then, all that needs to be done, for example, according to FIG. 6, is to attach a counter anchor 12 to the end of the pulling element 8 facing away from the anchor 7, wherein this counter anchor 12 can also be a stitch of the pulling element 8, in order to fix the housing 4 of the sensor 3, for example, within the heart wall 5.

According to FIGS. 7 to 10, the housing 4, which contains the sensor 3, has an approximately cylindrical cross-sectional form with two rounded ends, which simplifies the insertion of the housing 4 into an opening in the heart created by the puncturing cannula 10. If necessary, these ends could also be pointed. According to FIGS. 8 and 10, the pulling element 8 attaches to the two ends of the housing 4, so that it can be placed, for example, according to FIG. 11.

FIGS. 7 and 9 show a modified embodiment, which provides that the housing 4 of the sensor or sensors 3 has a continuous opening 13, which runs in its longitudinal direction and with which the housing 4 can be moved by means of the thread or pulling element 8 of the anchor 7, so that the sensor 3, after installation of the anchor 7 according to FIG. 5, can be moved by means of this thread 8 into its position of use with the help of a catheter 14.

Thus, it is possible either to bring the housing 4 with the sensor 3 into its position of use directly through the installation of the anchor 7 or else first to fix the anchor 7 with its pulling element 8 or thread to the heart wall 5 and then to move a sensor 3 with the help of a housing 4, which has an elongated opening 13, by means of the thread 8, which is used for holding and which simultaneously has the function of a guide thread or guide wire if the pulling element 8 is composed of wire. In this case, after the housing 4 is moved by this pulling element 8 into its position of use, on the outside of the heart a counter holder 12 can be provided through stitching, because the sensor is fixed sufficiently through friction, whether it is on the pulling element 8 or in the tissue of the heart 2.

In all of the embodiments, the housing 4 also has a thread running opposite the direction of insertion or a pulling element 15, and one can see in FIGS. 7 and 9 that the sensor 3 or its housing that can be pushed and moved on the pulling element 8 carries such an additional thread 15, with which the housing 4 can also be positioned opposite the direction of pushing or can be pulled out again from the heart 2. For example, if the housing 4 is pushed too far during the implantation with the help of the catheter 14, through a tensile force on this additional pulling element or thread 15, the housing 4 can be moved backwards again somewhat.

In contrast, in the embodiment according to FIGS. 8 and 10, the pulling element 8 with the anchor 7 attaches to one side of the sensor or its housing 4 and the pulling element 15 for the counter anchor or stitch attaches to the opposite side.

Here, the housing 4 of the sensor 3 has a relatively small cross section in relation to its length, in order to be able to be pushed easily into the heart tissue via a punctured channel and/or a pulling element 8. Simultaneously, the housing 4 is composed of biocompatible material.

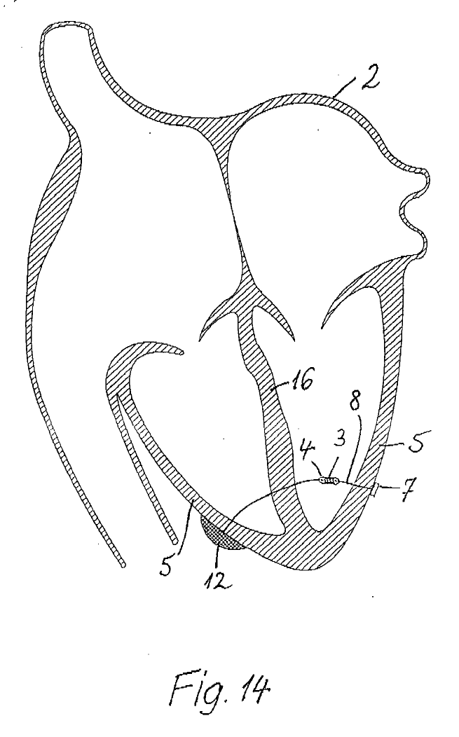

While a sensor 3 is arranged in the housing 4 in the embodiments according to FIGS. 7 and 8, the embodiments according to FIGS. 9 and 10 show that in the housing 4 there can also be at least two pressure detectors or sensors 3, which are arranged at a distance to each other or opposite each other. One such embodiment is especially well suited for implantation in the intermediate wall 16 of the heart, as shown in FIG. 12, so that the sensor facing the right ventricle can measure or monitor the pressure in the right ventricle and the other sensor 3 can measure or monitor the pressure in the left ventricle.

In contrast, FIG. 11 shows an arrangement, in which, in each of the outer walls 5 of the two ventricles, a housing 4 is implanted and fixed with the help of a pulling element 8 and an anchor 7, as well as a common counter anchor 12.

According to FIG. 13, in an analogous anchoring, as can be seen in FIG. 11, the two sensors 3 in the two ventricles can also be held in these ventricles 3, because, in this case, the pulling elements 8 penetrate through the ventricles to a certain extent.

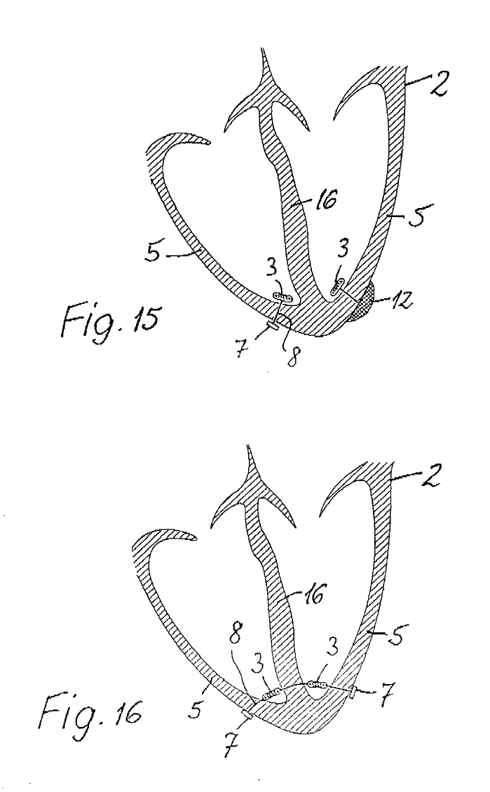

FIG. 14 shows a comparable arrangement with only one sensor 3 in the left ventricle, in which the pulling elements 8 and 15 penetrate both ventricles and are each fixed on the outside with an anchor 7 and a counter anchor 12, but this pressure sensor is arranged only in the region of the left ventricle.

FIG. 15 shows, in each ventricle, a sensor or a housing 4, wherein pulling elements penetrate the outer wall 5 of the heart and are fixed there with an anchor 7 or a counter anchor 12. A pulling element initially running between two housings 4 can be composed of a self-dissolving material and therefore is not seen in FIG. 15.

FIG. 16 also shows an arrangement with a pulling element, which runs through both ventricles and which is fixed on the outside with anchors 7, with a housing 4 with at least one sensor being arranged on the pulling element 8 in each ventricle.

Other arrangements and placements of even more such housings 4 with one or two or even more sensors 3 are possible due to the holder, which only slightly negatively affects the heart 2, and the very simple implantation method.

The device 1 that can be implanted from the outside under the outer side of a heart 2 for measuring biometric blood parameters, such as, for example, blood pressure, has at least one sensor 3, which can be analyzed telemetrically or by means of transponder technology and which is arranged on or in a housing 4, wherein a holder for fixing the sensor 3 or its housing 4, for example, in the interior of a heart wall also belongs to the device 1. A pin-like anchor 7, which can be inserted from the outside of the heart into or through the outer wall 5 of the heart 2 and/or the intermediate wall 6 and which is fixed to a pulling element 8 or thread that is connected in the position of use to the sensor 3 or to a housing 4 in a suitable way directly or via an opening 13 running in the longitudinal direction, is used as the holder. The anchor is formed so that it is held on one side in a puncturing cannula 10 and can be pushed or inserted on the other side with the help of a tool 9 or needle relative to this puncturing cannula 10 and/or through the heart wall 5. In the position of use, there is a counter anchor 12, for example, in a compact form or provided as a stitch, on the end of the pulling element 15 attaching to the opposite end of the housing 4.

It should also be mentioned that the counter anchor 12 can also contain a part of the electronics of the sensor 3 in the compact configuration shown in FIGS. 6 and 11 to 15 and then can be formed, for example, like a housing.

1. Device (1) for implantation under an upper or outer surface of a heart (2) for measuring biometric blood parameters, such as blood pressure, blood composition, pH value of the blood, blood-sugar level, temperature, comprising at least one sensor (3) that can be analyzed telemetrically or by transponder technology and with a holder for fixing the sensor (3) or a housing (4) containing the sensor on or in the heart (2), a pin-like anchor (7), which can be inserted from the outside of the heart (2) into or through an outer wall (5) of the heart (2) and/or an intermediate wall (6) and which is fixed to a pulling element (8) or thread connected to the sensor (3) at least in a position of use, the pulling element or thread is provided as a holder for the sensor (3) or the housing (4), wherein the pulling element (8) or the thread attaches to the pin-like anchor (7) between two ends thereof, and the anchor (7) can be pushed or inserted from outside into or through the heart wall (5) by a tool (9) or needle.

2. Device according to claim 1, wherein the anchor (7) can pivot relative to the pulling element (8) about an attachment point to the pulling element.

3. Device according to claim 1, wherein a deformation, which can be coupled detachably to the tool (9) or needle, is provided on an end of the anchor opposite a piercing tip (7a) or piercing end thereof as an attachment point for the tool (9) or the needle to the anchor (7).

4. Device according to claim 3, wherein the anchor (7) has a reduced cross section at a back side thereof in the insertion direction and facing away from the tip (7a) or insertable end for one end of the tool (9) and/or a receptacle opening (11) located in the anchor that is open at the back side and closed at an opposite end for the tool (9) or the needle to be received in the receptacle opening, so that the anchor (7) can be moved into a position of use.

5. Device according to claim 1, further comprising a puncturing cannula (10), whose hollow interior corresponds approximately to an outer cross section of the anchor (7) at least at an outer or distal end (10a) thereof and which holds the anchor (7) at least partially, and the thread or the pulling element (8) extends on an outside of the puncturing cannula (10).

6. Device according to claim 1, wherein the housing (4) of the sensor (3) has an approximately cylindrical cross-sectional form with at least one, rounded and/or pointed end, and the pulling element (8) attaches to or near one or both ends of the housing (4) at least in a position of use.

7. Device according to claim 1, wherein the housing (4) of the sensor (3) or the sensor itself has an opening (13), which extends in a longitudinal direction and through which it can be moved via the thread or the pulling element (8) of the anchor (7), so that the sensor (3) can be moved into a position of use by the thread (8) after installation of the anchor (7).

8. Device according to claim 1, wherein an end of the pulling element (8) or thread projecting past the sensor on a side facing away from the anchor in the position of use is provided with a counter anchor or stitch to form a counter holder.

9. Device according claim 1, wherein the sensor (3) that is movable with the pulling element (8) carries an additional thread (15), by which the sensor can also be positioned opposite a direction of pushing or can be removed again from the heart (2).

10. Device according to claim 9, wherein the pulling element (8) with the anchor (7) attaches to one side of the sensor (3) and the pulling element (15) for a counter anchor (12) or stitch attaches to an opposite side.

11. Device according to claim 1, wherein the housing (4) of the sensor (3) is composed of biocompatible material and has a small cross section in relation to a length thereof.

12. Device according to claim 1, wherein in the housing (4) of the sensor (3) there are at least two pressure detectors (3), which are spaced apart from each other or which are arranged opposite each other in or on the housing (4).

13. Device according to claim 10, wherein the counter anchor (12) includes at least a portion of electronics of the sensor (3).

14. Device according to claim 1, wherein the housing (4) of the sensor (3) has an approximately cylindrical cross-sectional form with opposing rounded and/or pointed ends, and the pulling element (8) attaches to or near one or both ends of the housing (4) at least in a position of use.
